(19) United States
(12) Patent Application Publication
(10) Pub. No.: US 2004/0023840 A1
(43) Pub. Date: Feb. 5, 2004

(54) Combination of organic compounds

(76) Inventors: Marc De Gasparo, Rossemaison (CH); Kurt C. Graves, Chester, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/257,559

(22) PCT Filed: Apr. 10, 2001
(86) PCT No.: PCT/EP01/04115

(51) Int. Cl. A61K 31/00

(52) U.S. Cl. 514/1

(57) ABSTRACT

The invention relates to a combination of at least two therapeutic combination components selected from the group consisting of (i) an AT₁-receptor antagonist or an AT₁ receptor antagonist combined with a diuretic or, in each case, a pharmaceutically acceptable salt thereof, (ii) a HMG-Co-A reductase inhibitor or a pharmaceutically acceptable salt thereof and (iii) an ACE inhibitor or a pharmaceutically acceptable salt thereof for use in the prevention of, delay of progression of, treatment of selected diseases and conditions.

The invention relates to a combination of at least two therapeutic combination components selected from the group consisting of

(i) an AT₁-receptor antagonist or an AT₁ receptor antagonist combined with a diuretic or, in each case, a pharmaceutically acceptable salt thereof,

(ii) a HMG-Co-A reductase inhibitor or a pharmaceutically acceptable salt thereof and

(iii) an ACE inhibitor or a pharmaceutically acceptable salt thereof for use in the prevention of, delay of progression of, treatment of a disease or condition selected from the group consisting of hyperlipidaemia and dyslipidemia, atherosclerosis, insulin resistance and syndrome X, diabetes mellitus type 2, obesity, nephropathy, renal failure, e.g. chronic renal failure, hypothyroidism, survival post myocardial infarction (MI), coronary heart diseases, hypertension in the elderly, familial dyslipidemic hypertension, and remodeling following hypertension (antiproliferative effect of the combination), all these diseases or conditions associated with or without hypertension, and, furthermore, in the prevention of, delay of progression of, treatment of stroke, erectile dysfunction and vascular disease.

The invention furthermore relates to a pharmaceutical composition for the prevention of, delay of progression of, treatment of a disease or condition selected from the group consisting of hyperlipidaemia and dyslipidemia, atherosclerosis, insulin resistance and syndrome X, diabetes mellitus type 2, obesity, nephropathy, renal failure, e.g. chronic renal failure, hypothyroidism, survival post MI, coronary heart diseases, hypertension in the elderly, familial dyslipidemic hypertension, and remodeling following hypertension (antiproliferative effect of the combination), all these diseases or conditions associated with or without hypertension, and, furthermore, for the prevention of, delay of progression of, treatment of stroke, erectile dysfunction and vascular disease, comprising

(a) a combination of at least two therapeutic combination components selected from the group consisting of

(i) an AT₁-receptor antagonist or an AT₁ receptor antagonist combined with a diuretic or, in each case, a pharmaceutically acceptable salt thereof,

(ii) a HMG-Co-A reductase inhibitor or a pharmaceutically acceptable salt thereof and

(iii) an ACE inhibitor or a pharmaceutically acceptable salt thereof, and

(b) a carrier.

The invention furthermore relates to a method of prevention of, delay of progression of or treatment of endothelial dysfunction with or without hypertension comprising administering to a warm-blooded animal, including man, in need thereof an effective amount of an AT₁ receptor antagonist or a pharmaceutically acceptable salt thereof or of a combination of an AT₁ receptor antagonist and an diuretic or a pharmaceutically acceptable salt thereof.

The invention furthermore relates to a method of prevention of, delay of progression of or treatment of endothelial dysfunction with or without hypertension comprising administering to a warm-blooded animal, including man, in need thereof an effective amount of a HMG-Co-A reductase inhibitor or a pharmaceutically acceptable salt thereof.

The invention furthermore relates to a method of prevention of, delay of progression of or treatment of endothelial dysfunction with or without hypertension comprising administering to a warm-blooded animal, including man, in need thereof an effective amount of an ACE inhibitor or a pharmaceutically acceptable salt thereof.

The invention furthermore relates to a method of prevention of, delay of progression of or treatment of endothelial dysfunction with or without hypertension comprising administering to a warm-blooded animal, including man, in need thereof a pharmaceutical composition comprising

a combination of at least two therapeutic agents selected from the group consisting of

(i) an AT₁-receptor antagonist or a pharmaceutically acceptable salt thereof,

(ii) a HMG-Co-A reductase inhibitor or a pharmaceutically acceptable salt thereof and

(iii) an ACEI inhibitor or a pharmaceutically acceptable salt thereof.

The invention furthermore relates to the use of

(a) either of

(i) an AT₁-receptor antagonist or a pharmaceutically acceptable salt thereof,

(ii) a HMG-Co-A reductase inhibitor or a pharmaceutically acceptable salt thereof or

(iii) an ACEI inhibitor or a pharmaceutically acceptable salt ther of; or

(b) a combination of

(i) an AT₁-receptor antagonist or a pharmaceutically acceptable salt thereof,

(ii) a HMG-Co-A reductase inhibitor or a pharmaceutically acceptable salt thereof or

(iii) an ACEI inhibitor or a pharmaceutically acceptable salt thereof for the manufacture of a medicament for the prevention of, delay of progression of, or treatment of

(α) a disease or condition selected from the group consisting of hyperlipidaemia and dyslipidemia, atherosclerosis, insulin resistance and syndrome X, diabetes mellitus type 2, obesity, nephropathy, renal failure, e.g. chronic renal failure, hypothyroidism, survival post MI, coronary heart diseases, hypertension in the elderly, familial dyslipidemic hypertension, and remodeling following hypertension (antiproliferative effect of the combination), all these diseases or conditions associated with or without hypertension; or

(β) endothelial dysfunction with or without hypertension; and

(γ) stroke, erectile dysfunction and vascular disease.

AT₁-receptor antagonists (also called angiotensin II receptor antagonists) are understood to be those active ingredients which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

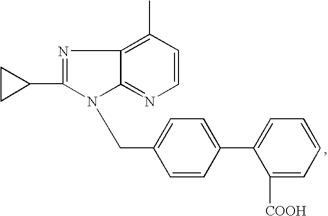

the compound with the designation SC-52458 of the following formula

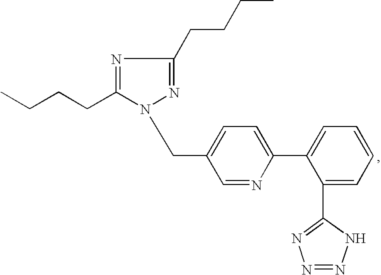

and the compound with the designation ZD-8731 of the following formula

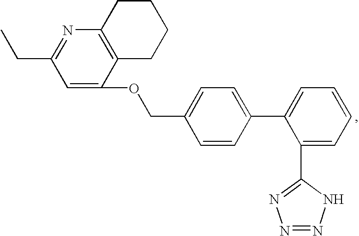

or, in each case, a pharmaceutically acceptable salt thereof.

Preferred AT₁-receptor antagonist are those agents which have been marketed, most preferred is valsartan or a pharmaceutically acceptable salt thereof.

A diuretic is, for example, a thiazide derivative selected from the group consisting of chlorothiazide, hydrochlorothiazide, methylclothiazide, and chlorothalidon. The most preferred is hydrochlorothiazide.

A preferred combination component “AT₁ receptor antagonist combined with a diuretic” is a combination of valsartan or losartan or, in each case, a pharmaceutically acceptable salt thereof and hydrochlorothiazide.

HMG-Co-A reductase inhibitors (also called β-hydroxy-β-methylglutaryl-co-enzyme-A reductase inhibitors) are understood to be those active agents which may be used to lower the lipid levels including cholesterol in blood.

The class of HMG-Co-A reductase inhibitors comprises compounds having differing structural features. For example, mention may be made of the compounds which are selected from the group consisting of atorvastatin, cerivastatin, fluvastatin, lovastatin, pitavastatin (formerly itavastatin), pravastatin, rosuvastatin, and simvastatin, or, in each case, a pharmaceutically acceptable salt thereof.

Preferred HMG-Co-A reductase inhibitors are those agents which have been marketed, most preferred is fluvastatin, atorvastatin, pitavastatin or simvastatin or a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents which have been marketed, most preferred are benazepril and enalapril.

A preferred composition comprises the combination of (i) the AT₁ receptor antagonist valsartan or a pharmaceutically acceptable salt thereof and (ii) a HMG-Co-A reductase inhibitor selected from the group consisting of fluvastatin, atorvastatin, pitavastatin and simvastatin or, in each case, a pharmaceutically acceptable salt thereof. Most preferred is the composition comprising (i) valsartan or a pharmaceutically acceptable salt thereof and (ii) pitavastatin or simvastatin or, in each case, a pharmaceutically acceptable salt thereof. Likewise preferred is a corresponding composition where valsartan is replaced with a combination of valsartan with hydrochlorothiazide.

A preferred composition comprises the combination of (i) the AT₁ receptor antagonist valsartan or a pharmaceutically acceptable salt thereof and (ii) the ACE inhibitor benazepril or enalapril or, in each case, a pharmaceutically accetable salt thereof.

A preferred composition comprises the combination of (i) a HMG-Co-A reductase inhibitor selected from the group consisting of fluvastatin, atorvastatin, pitavastatin and simvastatin or, in each case, a pharmaceutically acceptable salt thereof and (ii) the ACE inhibitor benazepril or enalapril or, in each case, a pharmaceutically accetable salt thereof. Most preferred is the composition comprising (i) pitavastatin or simvastatin or, in each case, a pharmaceutically acceptable salt thereof and (ii) benazepril or enalapril or, in each case, a pharmaceutically acceptable salt thereof. Likewise preferred is a corresponding composition where valsartan is replaced with a combination of valsartan with hydrochlorothiazide.

The structure of the active agents identified hereinbefore or hereinafter by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The corresponding active ingredients or a pharmaceutically acceptable salts thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

The pharmaceutical activities as effected by administration of representatives of the class of AT₁-receptor antagonists or ACE inhibitors, respectively, or of the combination of active agents used according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

Endothelial dysfunction is being acknowledged as a critical factor in vascular diseases. The endothelium plays a bimodal role as the source of various hormones or by-products with opposing effects: vasodilation and vasoconstriction, inhibition or promotion of growth, fibrinolysis or thrombogenesis, production of anti-oxidants or oxidising agents. Genetically predisposed hypertensive animals with endothelial dysfunction constitute a valid model for assessing the efficacy of a cardiovascular therapy.

Endothelial disfunction is characterized by, for example, increased oxidative stress, causing decreased nitric oxide, increased factors involved in coagulation or fibrinolysis such as plasminogen activating inhibitor-1 (PAI-1), tissue factor (TF), tissue plasminogen activator (tPA), increased adhesion molecules such as ICAM and VCAM, increased growth factors such as bFGF, TGFb, PDGF, VEGF, all factors causing cell growth inflammation and fibrosis.

The treatment e.g. of endothelial dysfunction can be demonstrated in the following pharmacological test:

### Material and Methods

Male 20-24 week-old SHR, purchased from RCC Ldt (Fullingsdorf, Switzerland), are maintained in a temperature- and light-controlled room with free access to rat chow (Nafag 9331, Gossau, Switzerland) and tap water. The experiment is p rformed in accordance with the NIH guidelines and approved by the Canton Veterinary office (Bew 161, Kantonales V t rin\overline{a}ramt, Liestal, Switzerland). All rats are treated with the NO synthase inhibitor L-NAME (Sigma Chemicals) administered in drinking water (50 mg/l) for 12 weeks. The average daily dose of L-NAME calculated from the water consumed was 2.5 mg/kg/d (range 2.1-2.7).

The rats are divided into 5 groups: group 1, control (n=40); Group 2, valsartan (val5, 5 mg/kg/d; n=40); Group 3, enalapril (ena1, 1 mg/kg/d; n=30); Group 4, a combination (ena1val5) of enalapril (1 mg/kg/d) and valsartan (5 mg/kg/d); n=30) and Group 5, valsartan (val50, 50 mg/kg/d; n=30). The drugs are administered in drinking fluid. The dose of enalapril was selected from the work of Sweet et al. (1987) indicating significantly increased survival in rats with healed myocardial infarction. The pressor effect of Ang II at 1 mg/kg obtained in controls normotensive rats is reduced by 49% and 73% after treatment with valsartan 5 and 50 mg/kg/d, respectively (Gervais et al. 1999). The response to Ang I injected in Wistar Kyoto rats pretreated with enalapril 1 mg/kg/d or valsartan 5 mg/kg/d is similar.

Body weight is measured every week. Systolic blood pressure and heart rate are recorded by tail cuff plethysmography 3 and 2 weeks before starting the study and at 2 weeks after drug administration. Urine is collected over a 24 hour period from rats kept in individual (metabolic) cages the week before starting treatment and at weeks 4 and 12 for volume measurement and protein, creatinine, sodium and potassium determination using standard laboratory methods. At the same time points, blood samples are withdrawn from the retro-orbital plexus (maximum 1 ml) for creatinine, Na⁺ and K⁺ assays.

Ten rats from each group are sacrificed at 4 weeks for collection of kidney and heart for morphological analysis. The remaining rats are sacrificed at 12 weeks. Cardiac and kidney weight is recorded. Terminal blood sampling is performed in 5% EDTA at 4 (morphometry study) and 12 (end of the study) weeks for aldosterone, determination by radioimmunoassay using a DPC coat-a-count aldosterone-RIA kit (Bühlmann, Switzerland).

### Statistical Analysis

All data are expressed as mean±SEM. Statistical analysis is performed using a one-way ANOVA, followed by a Duncan's multiple range test and a Newman-Keuls test, 7 for comparison between the different groups. Results with a probability value of less than 0.05 are deemed statistically significant.

### Results

Even at non-blood pressure reducing doses, both valsartan and enalapril treatment led to significant improvements in survival rates (67% and 55%, respectively). Combining the AT₁-receptor blocker and the ACE inhibitor led to an even more dramatic increase in survival rate to 85%. Again, this benefit occurred without affecting blood pressure, which remained around 275 mmHg. A high dose of valsartan (50 mg/kg) which significantly attenuated the increase in blood pressure (systolic blood pressure above 250 mmHg), led to a 95% survival rate. Untreated animals with chronic NO synthase blockade had a mortality rate of 63% within 12 weeks.

In untreated animals, the high mortality can be attributed principally to the development of malignant hypertension and endothelial dysfunction. The more than additive effects on survival from AT₁-receptor blocker and the ACE inhibitor in non-hypotensive doses might be related to a more complete blockade of the tissue RAS, independent of any effect on blood pressure.

The surprising observation is that, in this model, blockade of the RAS with low doses of valsartan and enalapril improved survival despite persistent kidney dysfunction and high blood pressure. There was no decrease in proteinuria and no reduction of kidney lesions. Kidney and heart sections showed glomeruloslerosis, fibrinoid necrosis and fibrosis. These results clearly demonstrate that survival of SHR with endothelial dysfunction is independent of the blood-pressure lowering effect of the treatment and may be related to a direct effect on the endothelium.

An improvement of regression of atherosclerosis without effecting the serum lipid levels can, for exmple, be demonstrated by using the animal model as disclosed by H. Kano et al. in Biochemical and Biophysical Research Communications 259, 414-419 (1999).

That the compounds or combinations according to the present invention can be used for the regression of a cholesterol diet-induced atherosclerosis, can be demonstrated using the test model described, e.g., by C. Jiang et al. in Br. J. Pharmacol. (1991), 104, 1033-1037.

That the compounds or combinations according to the present invention can be used for the treatment of renal failure, especially chronic renal failure, can be demonstrated using the test model described, e.g., by D. Cohen et al. in Journal of Cardiovascular Pharmacology, 32: 87-95 (1998).

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

All the more surprising is the experimental finding that the combined administration of combination according to the present invention results in a beneficial, especially a synergistic (=more than additive effect), therapeutic effect, furthermore, in benefits resulting from the combined treatment and further surprising beneficial effects compared to a monotherapy applying only one of the pharmaceutically active compounds used in the combinations disclosed herein.

In particular, all the more surprising is the experimental finding that the combination of the present invention results in a beneficial, especially a synergistic, therapeutic effect but also in benefits resulting from combined treatment such as a surprising prolongation of efficacy, a broader variety of therapeutic treatment and surprising beneficial effects on diseases and conditions as specified hereinbefore or hereinafter.

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

Preferably, the jointly therapeutically effective amounts of the active agents according to the combination of the present invention can be administered simultaneously or sequentially in any order, separately or in a fixed combination.

The pharmaceutical composition according to the present invention as described hereinbefore and hereinafter may be used for simultaneous use or sequential use in any order, for separate use or as a fixed combination.

he present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The invention furthermore relates to a commercial package comprising the combination according to the present invention together with instructions for simultaneous, separate or sequential use.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commerically available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

Valsartan, as a representative of the class of AT₁-receptor antagonists, will be supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting e.g. with a daily dose of 20 mg or 40 mg of valsartan, increasing via 80 mg daily and further to 160 mg daily up to 320 mg daily. Preferably, valsartan is applied twice a day with a dose of 80 mg or 160 mg, respectively, each. Corresponding doses may be taken, for example, in the moming, at mid-day or in the evening.

In case of HMG-Co-A reductase inhibitors, preferred dosage unit forms of HMG-Co-A reductase inhibitors are, for example, tablets or capsules comprising e.g. from about 5 mg to about 120 mg, preferably, when using fluvastatin, for example, 20 mg, 40 mg or 80 mg (equivalent to the free acid) of fluvastatin, for example, administered once a day.

In case of ACE inhibitors, preferred dosage unit forms of ACE inhibitors are, for example, tablets or capsules comprising e.g. from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is t.i.d. administration.

Especially preferred are low dose combinations.

The following examples illustrate the above-described invention; however, it is not intended to restrict the scope of this invention in any manner.

### FORMULATION EXAMPLE 1

Film-Coated Tablets:

| Components | Composition Per Unit (mg) | Standards |
|---|---|---|
| Granulation | | |
| Valsartan [= active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/ | 0.75 | Ph. Eur/ |
| colloidal silicon dioxide/Aerosil 200 | | NF |
| Magnesium stearate | 2.5 | NF, Ph. Eur |
| Blending | | |
| Colloidal anhydrous silica/ | 0.75 | Ph. Eur/ |
| colloidal silicorr dioxide/Aerosil 200 | | NF |
| Magnesium stearate | 2.00 | NF, Ph. Eur |
| Coating | | |
| Purified water^{*)} | — | |
| DIOLACK pale red 00F34899 | 7.00 | |
| Total tablet mass | 167.00 | |

^{*)}Removed during processing.

The film-coated tablet is manufactured e.g. as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screnning mill. The resulting mixture is again pre-mixed in a diffusion mixer, compacted in a roller compacter and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tabletts are coated with a film by using Diolack pale red in a perforated pan.

### FORMULATION EXAMPLE 2

Film-Coated Tablets:

| Components | Composition Per Unit (mg) | Standards |
|---|---|---|
| Granulation | | |
| Valsartan [= active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/ | 1.50 | Ph. Eur/ |
| colloidal silicon dioxide/Aerosil 200 | | NF |
| Magnesium stearate | 5.00 | NF, Ph. Eur |
| Blending | | |
| Colloidal anhydrous silica/ | 1.50 | Ph. Eur/ |
| colloidal silicon dioxide/Aerosil 200 | | NF |
| Magnesium stearate | 4.00 | NF, Ph. Eur |
| Coating | | |
| Opadry Light Brown 00F33172 | 10.00 | |
| Total tablet mass | 330.00 | |

The film-coated tablet is manufactured e.g. as described in Formulation Example 1.

### FORMULATION EXAMPLE 3

Film-Coated Tablets:

| Components | Composition Per Unit (mg) | Standards |
|---|---|---|
| Core: Internal phase | | |
| Valsartan | 40.00 | |
| [= active ingredient] | | |
| Silica, colloidal anhydrous | 1.00 | Ph. Eur, USP/NF |
| (Colloidal silicon dioxide) | | |
| [= Glidant] | | |
| Magnesium stearate | 2.00 | USP/NF |
| [= Lubricant] | | |
| Crospovidone | 20.00 | Ph. Eur |
| [Disintegrant] | | |
| Microcrystalline cellulose | 124.00 | USP/NF |
| [= Binding agent] | | |
| External phase | | |
| Silica, colloidal anhydrous, | 1.00 | Ph. Eur, USP/NF |
| (Colloidal silicon dioxide) | | |
| [= Glidant] | | |
| Magnesium stearate | 2.00 | USP/NF |
| [Lubricant] | | |
| Film coating | | |
| Opadry ® brown OOF 16711^{*)} | 9.40 | |
| Purified Water^{**)} | — | |
| Total tablet mass | 199.44 | |

^{*)}The composition of the Opadry ® brown OOF16711 coloring agent is tabulated below.
^{**)}Removed during processing

Opadry ® Composition:

| Ingredient | Approximate % Composition |
|---|---|
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablet is manufactured e.g. as described in Formulation Example 1.

### FORMULATION EXAMPLE 4

Capsules:

| Components | Composition Per Unit (mg) |
|---|---|
| Valsartan [= active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total tablet mass | 209.50 |

The tablet is manufactured e.g. as follows:

### Granulation/Drying

Valsartan and microcrystallin cellulose are spray-granulated in a fluidised bed granulator with a granulating solution consisting of povidone and sodium lauryl sulphate dissolved in purified water. The granulate obtained is dried in a fluidiesd bed dryer.

### Milling/Blending

The dried granulate is milled together with crospovidone and magnesium stearate. The mass is then blended in a conical srew type mixer for approximately 10 minutes.

### Encapsulation

Teh empty hard gelatin capsules are filled with the blended bulk granules under controlled temperature and humidity conditions. The filed capsules are dedustee, visually inspected, weightchecked and quarantied until by Quality assurance department.

### FORMULATION EXAMPLE 5

Capsules:

| Components | Composition Per Unit (mg) |
|---|---|
| Valsartan [= active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total tablet mass | 342.00 |

The formulation is manufactured e.g. as described in Formulation Example 4.

### FORMULATION EXAMPLE 6

Hard Gelatine Capsule:

| Components | Composition Per Unit (mg) |
|---|---|
| Valsartan [= active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| Total tablet mass | 130.00 |

### EXAMPLES 7 TO 11

| | 7 | 8 | 9 | 10 | 11 |
|---|---|---|---|---|---|
| | Amount | Amount | Amount | Amount | Amount |
| Example | per Unit | per Unit | per Unit | per Unit | per Unit |
| Components | (mg) | (mg) | (mg) | (mg) | (mg) |
| Granulation | | | | | |
| Valsartan Drug Substance | 80.000 | 160.000 | 40.000 | 320.000 | 320.000 |
| Microcrystalline Cellulose (NF, | 54.000 | 108.000 | 27.000 | 216.000 | 216.000 |
| Ph.Eur.)/Avicel PH 102 | | | | | |
| Crospovidone (NF, Ph.Eur.) | 15.000 | 30.000 | 7.500 | 80.000 | 60.000 |
| Colloidal Anhydrous Silica (Ph. | 1.500 | 3.000 | 0.750 | 3.000 | 6.000 |
| Eur.)/Colloidal Silicon Dioxide | | | | | |
| (NF)/Aerosil 200 | | | | | |
| Magnesium Stearate (NF, | 3.000 | 6.000 | 1.500 | 10.000 | 12.000 |
| Ph.Eur.) | | | | | |
| Blending | | | | | |
| Colloidal Anhydrous Silica (Ph. | — | — | — | 3.000 | — |
| Eur.)/Colloidal Silicon Dioxide | | | | | |
| (NF)/Aerosil 200 | | | | | |
| Magnesium Stearate, NF, | 1.500 | 3.000 | 0.750 | 8.000 | 6.000 |
| Ph.Eur. | | | | | |
| Core Weight/mg | 155.000 | 310.000 | 77.500 | 640.000 | 620.000 |
| Coating | — | — | 3.800 | 15.000 | 16.000 |

### EXAMPLE 12

Hard gelatin capsule:

| Component | Amount per unit [mg] |
|---|---|
| Capsule | |
| Fluvastatin Sodium¹⁾ | 21.481²⁾ |
| Calcium Carbonate | 62.840 |
| Sodium Bicarbonate | 2.000 |
| Microcrystalline Cellulose | 57.220 |
| Pregelatinized Starch | 41.900 |
| Purified Water³⁾ | Q.S. |
| Magnesium Stearate | 1.050 |
| Talc | 9.430 |
| Target Capsule Fill Weight | 195.92 |
| Capsule Shell | |
| Hard gelatin Capsule Shell | 48.500 |
| Branding Ink (pre-printed) | |
| White Ink | Trace |
| Red Ink | Trace |
| Target Capsule Weight | 244.42 |

¹⁾includes a 2% overage for moisture
²⁾20 mg of free acid is equivalent to 21.06 mg Na salt
³⁾partially removed during processing

### EXAMPLE 13

Hard gelatin capsule

| Component | Amount per unit [mg] |
|---|---|
| Capsule | |
| Fluvastatin Sodium | 42.962¹⁾²⁾ |
| Calcium Carbonate | 125.680 |
| Sodium Bicarbonate | 4.000 |
| Microcrystalline Cellulose | 114.440 |
| Pregelatinized Starch | 83.800 |
| Purified Water³⁾ | Q.S. |
| Magnesium Stearate | 2.100 |
| Talc | 18.860 |
| Target Capsule Fill Weight | 391.840 |
| Capsule Shell | |
| Hard gelatin Capsule Shell | 76.500 |
| Branding Ink (pre-printed) | |
| White Ink | Trace |
| Red Ink | Trace |
| Target Capsule Weight | 468.34 |

¹⁾includes a 2% overage for moisture
²⁾20 mg of free acid is equivalent to 21.06 mg Na salt
³⁾partially removed during processing

### EXAMPLE 14

Round, slightly bi-convex, film-coated tablets with beleved edges:

| Component | Amount per unit [mg] |
|---|---|
| Table Core | |
| Fluvastatin Sodium¹⁾ | 84.24²⁾ |
| Cellulose Microcrystalline/Micro- | 111.27 |
| crystalline cellulose fine powder | |
| Hypromellose/Hydroxypropyl | 97.50 |
| methyl cellulose (Methocel | |
| K100LVP CR; HPMC100 cps) | |
| Hydroxypropyl cellulose (Klucel | 16.25 |
| HXF) | |
| Potassium hydrogen carbonate/ | 8.42 |
| Potassium bicarbonate | |
| Povidone | 4.88 |
| Magnesium stearate | 2.44 |
| Core Tablet Weight | 325.00 |
| Coating | |
| Coating premix - Opadry Yellow | 9.75 |
| (00F22737) | |
| Total Weight | 334.75 |
| Water, purified³⁾ | Q.S. |

¹⁾84.24 mg of the sodium salt of fluvastatin is equivalent to 80 mg of fluvastatin free acid
²⁾to be adjusted for moisture (LOD)
³⁾removed during processing

### EXAMPLE 15

Round, biconvex, beveled-edged, film-coated tablets

| | Unit wt./Vol. | Unit wt./Vol. | Unit wt./Vol. | Unit wt./Vol. |
|---|---|---|---|---|
| Component | [mg] | [mg] | [mg] | [mg] |
| Benazepril Hydrochloride | 5.00 | 10.00 | 20.00 | 40.00 |
| Lactose Monohydrate, NF | 142.00 | 132.00 | 117.00 | 97.00 |
| Pregelatinized Starch, NF | 8.00 | 8.00 | 8.00 | 8.00 |
| Colloidial Silicon Dioxide, NF | 1.00 | 1.00 | 1.00 | 1.00 |
| (Cab-O-Sil, M-5) | | | | |
| Crospovidone, NF | 3.00 | 3.00 | 3.00 | 3.00 |
| Microcrystalline Cellulose, NF | 18.00 | 18.00 | 18.00 | 24.25 |
| Hydrogenated Castor Oil, NF | 8.00 | 8.00 | | |
| Magnesium Stearate, NF | | | 8.00 | 1.75 |
| Color: | — | | | 0.50 |
| Yellow-Brown (suspension) | | 2.00 | | |
| Red-Brown (suspension) | | | 0.50 | |
| Purified Water, USP | trace | trace | trace | trace |
| Opadry Color: | | | | |
| Yellow | 8.38 | 8.38 | | |
| Pink | | | 8.38 | 8.38 |
| Total | 193.38 | 190.38 | 183.88 | 183.88 |

What is claimed is:

1. Use of a combination of at least two therapeutic combination components selected from the group consisting of
(i) an AT₁-receptor antagonist or an AT₁ receptor antagonist combined with a diuretic or, in each case, a pharmaceutically acceptable salt thereof,
(ii) a HMG-Co-A reductase inhibitor or a pharmaceutically acceptable salt thereof and
(iii) an ACE inhibitor or a pharmaceutically acceptable salt thereof; for the manufacture of a medicament for the prevention of, delay of progression of, treatment of a disease or condition selected from the group consisting of hyperlipidaemia and dyslipidemia, atherosclerosis, insulin resistance and syndrome X, diabetes mellitus type 2, obesity, nephropathy, renal failure, hypothyroidism, survival post MI, coronary heart diseases, hypertension in the elderly, familial dyslipidemic hypertension, and remodeling following hypertension (antiproliferative effect of the combination), all these diseases or conditions associated with or without hypertension, and, furthermore, for the prevention of, delay of progression of, treatment of stroke, erectile dysfunction and vascular disease.

2. Use according to claim 1 wherein said AT₁-receptor antagionist is selected from the group consisting of valsartan, losartan, candesartan, eprosartan, irbesartan, saprisartan, tasosartan, telmisartan, the compound with the designation E-1477 of the following formula 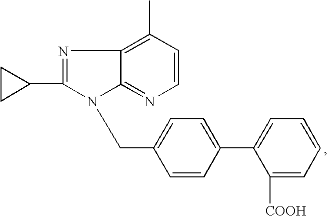
the compound with the designation SC-52458 of the following formula 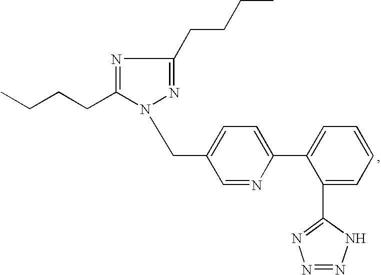
and the compound with the designation ZD-8731 of the following formula 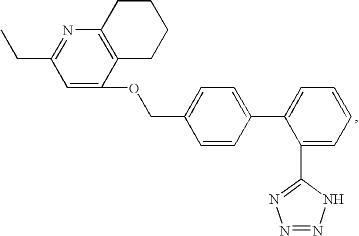
or, in each case, a pharmaceutically acceptable salt thereof.

3. Use according to claim 2 wherein said AT₁-receptor antagonist is valsartan or a pharmaceutically acceptable salt thereof.

4. Use according to any one of claims 1 to 3 wherein said HMG-Co-A reductase inhibitor is selected from the group consisting of atorvastatin, cerivastatin, fluvastatin, lovastatin, pitavastatin, pravastatin, rosuvastafin, and simvastatin, or, in each case, a pharmaceutically acceptable salt thereof.

5. Use according to claim 4 wherein said HMG-Co-A reductase inhibitor is fluvastatin, atorvastafin, pitavastatin or simvastatin.

6. Use according to any one of claims 1 to 5 wherein said ACE inhibitor is selected from th group consisting of alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

7. Use according to claim 6 wherein said ACE inhibitor is benazepril or enalapril or a pharmaceutically acceptable salt thereof.

8. Use of a therapeutic agent selected from the group consisting of
(i) an AT₁-receptor antagonist or an AT₁ receptor antagonist combined with a diuretic or, in each case, a pharmaceutically acceptable salt thereof,
(ii) a HMG-Co-A reductase inhibitor or a pharmaceutically acceptable salt thereof and
(iii) an ACE inhibitor or a pharmaceutically acceptable salt thereof, for the manufacture of a medicament for the prevention of, delay of progression of or treatment of endothelial dysfunction with or without hypertension.

9. A pharmaceutical composition for the prevention of, delay of progression of, treatment of a disease or condition selected from the group consisting of hyperlipidaemia and dyslipidemia, atherosclerosis, insulin resistance and syndrome X, diabetes mellitus type 2, obesity, nephropathy, renal failure, hypothyroidism, survival post MI, coronary heart diseases, hypertension in the elderly, familial dyslipidemic hypertension, and remodeling following hypertension (antiproliferative effect of the combination), all these diseases or conditions associated with or without hypertension, and, furthermore, in the prevention of, delay of progression of, treatment of stroke, erectile dysfunction and vascular disease, comprising
(a) a combination of at least two therapeutic combination components selected from the group consisting of
(i) an AT₁-receptor antagonist or an AT₁ receptor antagonist combined with a diuretic or, in each case, a pharmaceutically acceptable salt thereof,
(ii) a HMG-Co-A reductase inhibitor or a pharmaceutically acceptable salt thereof and
(iii) an ACE inhibitor or a pharmaceutically acceptable salt thereof, and
(b) a carrier.

10. A method of prevention of, delay of progression of or treatment of endothelial dysfunction with or without hypertension comprising administering to a warm-blooded animal, including man, in need thereof an effective amount of
(a) an AT₁ receptor antagonist or an AT₁ receptor antagonist combined with a diuretic or, in each case, a pharmaceutically acceptable salt thereof;
(b) a HMG-Co-A reductase inhibitor or a pharmaceutically acceptable salt thereof;
(c) an ACE inhibitor or a pharmaceutically acceptable salt thereof; or
(d) a combination of at least two therapeutic combination components selected from the group consisting of
(i) an AT₁-receptor antagonist or an AT₁ receptor antagonist combined with a diuretic or, in each case, a pharmaceutically acceptable salt thereof,
(ii) a HMG-Co-A reductase inhibitor or a pharmaceutically acceptable salt thereof and
(iii) an ACEI inhibitor or a pharmaceutically acceptable salt thereof.
